(19) United States
(12) Patent Application Publication
(10) Pub. No.: US 2016/0310627 A1
(43) Pub. Date: Oct. 27, 2016

(54) Air Purification Assembly

(71) Applicant: LaVance Frazier, Huntsville, AL (US)

(72) Inventor: LaVance Frazier, Huntsville, AL (US)

(21) Appl. No.: 14/692,904

(22) Filed: Apr. 22, 2015

(51) Int. Cl. A61L 9/20

(52) U.S. Cl. CPC A61L 9/20

(57) ABSTRACT

An air purification assembly for includes a housing that is structured to have an inlet opening and an outlet opening. The housing may be positioned proximate a return air duct of a forced air system. A purification unit is positioned within the housing and the purification unit is in fluid communication between the outlet opening and the inlet opening. The purification unit purifies air urged from the return air duct of the forced air system.

### BACKGROUND OF THE DISCLOSURE

### Field of the Disclosure

The disclosure relates to air purification devices and more particularly pertains to a new air purification device for removing odors from air urged through a return air duct of a forced air system.

### SUMMARY OF THE DISCLOSURE

An embodiment of the disclosure meets the needs presented above by generally comprising a housing that is structured to have an inlet opening and an outlet opening. The housing may be positioned proximate a return air duct of a forced air system. A purification unit is positioned within the housing and the purification unit is in fluid communication between the outlet opening and the inlet opening. The purification unit purifies air urged from the return air duct of the forced air system.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a front perspective view of a air purification assembly according to an embodiment of the disclosure.

FIG. 2 is a back view of an embodiment of the disclosure.

FIG. 3 is a front view of an embodiment of the disclosure.

FIG. 4 is a left side view of an embodiment of the disclosure.

FIG. 5 is a schematic view of an embodiment of the disclosure.

### DESCRIPTION OF THE PREFERRED EMBODIMENT

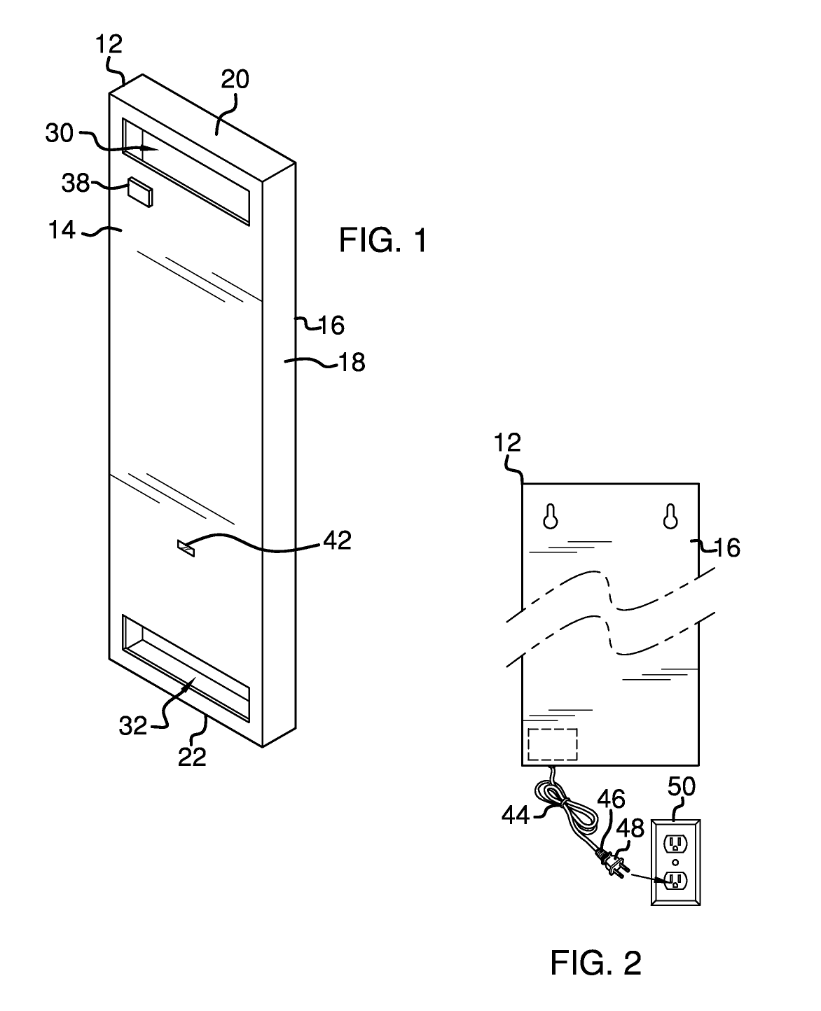

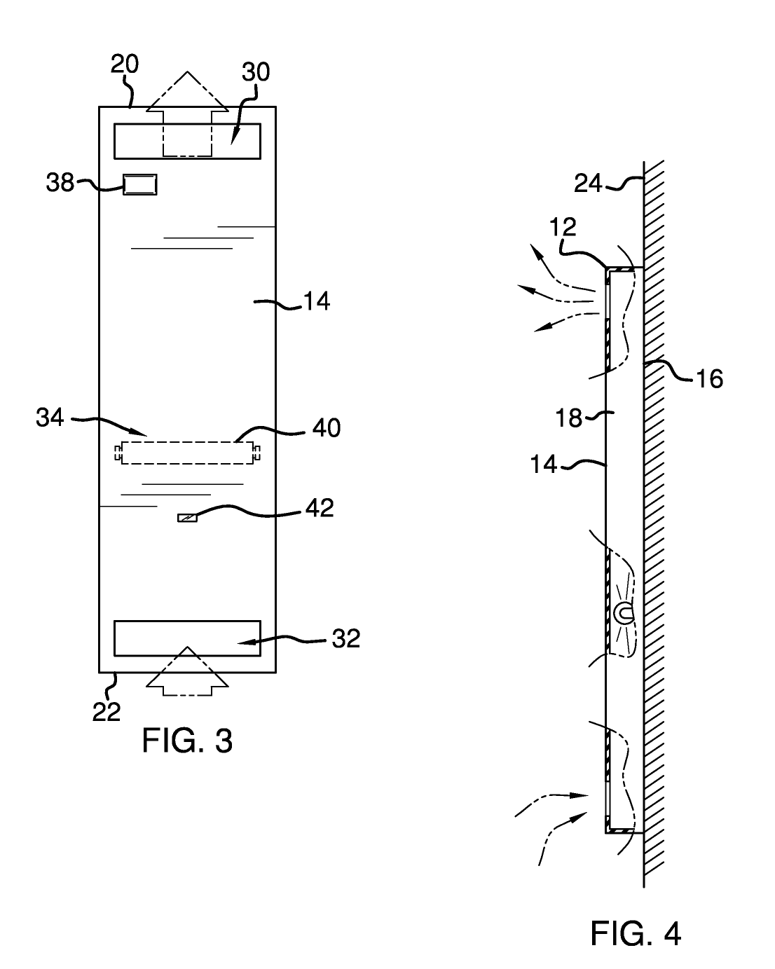

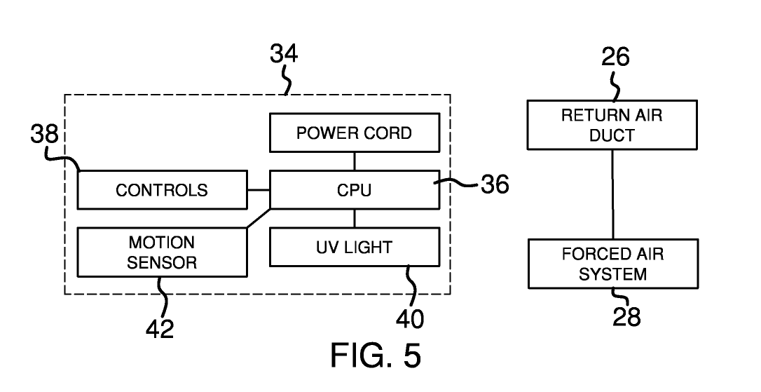

With reference now to the drawings, and in particular to FIGS. 1 through 5 thereof, a new air purification device embodying the principles and concepts of an embodiment of the disclosure and generally designated by the reference numeral 10 will be described.

As best illustrated in FIGS. 1 through 5, the air purification assembly 10 generally comprises a housing 12 that has a front wall 14, a back wall 16 and a peripheral wall 18 extending therebetween. The peripheral wall 18 has a top side 20 and a bottom side 22 and the back wall 16 may be coupled to a support surface 24. The support surface 24 may be a wall or the like and the housing 12 may be positioned proximate a return air duct 26 of a forced air system 28. The forced air system 28 may be a forced air system 28 of any conventional design. The front wall 14 has an outlet opening 30 extending therethrough and the outlet opening 30 is positioned adjacent to the top side 20. Additionally, the front wall 14 has an inlet opening 32 extending therethrough and the inlet opening 32 is positioned adjacent to the bottom side 22.

A purification unit 34 is positioned within the housing 12 and the purification unit 34 is in fluid communication between the outlet opening 30 and the inlet opening 32. The purification unit 34 purifies air urged from the return air duct 26 of the forced air system 28. The purification unit 34 comprises a control circuit 36 positioned within the housing 12. An actuator 38 is coupled to the housing 12 and the actuator 38 is electrically coupled to the control circuit 36. The actuator 38 is positioned on the front wall 14 adjacent to the top side 20 and the actuator 38 is actuatable between an on position and an off position.

A light emitter 40 is positioned within an interior of the housing 12 and the light emitter 40 is positioned closer to the inlet opening 32 than the outlet opening 30. The light emitter 40 is electrically coupled to the actuator 38 thereby facilitating the light emitter 40 being actuated and de-actuated by the actuator 38. The light emitter 40 emits electromagnetic radiation such that the light emitter 40 urges air inwardly through the inlet opening 32 and outwardly through the outlet opening 30 via thermal convection. The light emitter 40 eliminates bacteria in the air thereby facilitating odors being eliminated from the air moving from the inlet opening 32 to the outlet opening 30. The light emitter 40 may comprise an ultraviolet light emitter that emits short-wavelength (UV-C) ultraviolet radiation.

A motion sensor 42 is coupled to the housing 12 and the motion sensor 42 is positioned on the front wall 14 thereby facilitating the motion sensor 42 to detect motion proximate the housing 12. The motion sensor 42 is electrically coupled to the light emitter 40 and the motion sensor 42 de-actuates the light emitter 40 when the motion sensor 42 detects motion. The motion sensor 42 actuates the light emitter 40 when the motion sensor 42 ceases to detect motion. The motion sensor 42 prevents a person from being exposed to the radiation emitted by the light emitter 40.

A power cord 44 is electrically coupled to the actuator 38 and the power cord 44 extends outwardly from the bottom side 22. The power cord 44 has a distal end 46 with respect to the bottom side 22 and the distal end 46 has a plug 48 electrically coupled thereto. The plug 48 may be electrically coupled to a power source 50 and the power source 50 may be an electrical outlet or the like.

In use, the housing 12 is coupled to the support surface 24 and positioned proximate the return air duct 26. The actuator 38 is positioned in the on position to actuate the light emitter 40. The light emitter 40 draws air inwardly through the inlet opening 32 and urges the air outwardly through the outlet opening 30 via heat convection. The light emitter 40 eliminates bacteria in the air urged through the housing 12 in order to eliminate odors from the air.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. An air purification assembly configured to remove odors from and disinfect air urged by a forced air system, said assembly comprising:
a housing being structured to have an inlet opening and an outlet opening, said housing being configured to be positioned proximate a return air duct of a forced air system, said housing has a front wall and a top side; and
a purification unit positioned within said housing, said purification unit being in fluid communication with each of said outlet opening and said inlet opening, said purification unit being configured to purify air urged from the return air duct of the forced air system, said purification unit comprises a control circuit positioned within said housing, an actuator coupled to said housing, said actuator being electrically coupled to said control circuit, said actuator being positioned on said front wall adjacent to said top side, said actuator being actuatable between an on position and an off position; and
an ultraviolet light emitter positioned within an interior of said housing, said ultraviolet light emitter being positioned closer to said inlet opening than said outlet opening, said ultraviolet light emitter being electrically coupled to said actuator such that said ultraviolet light emitter is actuated and de-actuated by said actuator, said ultraviolet light emitter emitting electromagnetic radiation such that said ultraviolet light emitter is configured to urge air inwardly through said inlet opening and outwardly through said outlet opening via thermal convection, said ultraviolet light emitter being configured to eliminate bacteria in the air such that odors are eliminated from the air moving from said inlet opening to said outlet opening.

2. The assembly according to claim 1, wherein said housing has a front wall, a back wall and a peripheral wall extending therebetween, said peripheral wall having a top side and a bottom side, said back wall being configured to be coupled to a support surface, said front wall having said outlet opening extending therethrough, said outlet opening being positioned adjacent to said top side, said front wall having said inlet opening extending therethrough, said inlet opening being positioned adjacent to said bottom side.

3. (canceled)

4. (canceled)

5. The assembly according to claim 1, wherein a motion sensor coupled to said housing, said motion sensor being positioned on said front wall such that said motion sensor is configured to detect motion proximate said housing, said motion sensor being electrically coupled to said ultraviolet light emitter, said motion sensor de-actuating said ultraviolet light emitter when said motion sensor detects motion, said motion sensor actuating said ultraviolet light emitter when said motion sensor ceases to detect motion.

6. The assembly according to claim 5, wherein a power cord electrically coupled to said actuator, said power cord extending outwardly from said bottom side, said power cord having a distal end with respect to said bottom side, said distal end having a plug electrically coupled thereto, said plug being configured to be electrically coupled to a power source.

7. An air purification assembly configured to remove odors from and disinfect air urged by a forced air system, said assembly comprising:
a housing having a front wall, a back wall and a peripheral wall extending therebetween, said peripheral wall having a top side and a bottom side, said back wall being configured to be coupled to a support surface, said housing being configured to be positioned proximate a return air duct of a forced air system, said front wall having an outlet opening extending therethrough, said outlet opening being positioned adjacent to said top side, said front wall having an inlet opening extending therethrough, said inlet opening being positioned adjacent to said bottom side;
a purification unit positioned within said housing, said purification unit being in fluid communication with each of said outlet opening and said inlet opening, said purification unit being configured to purify air urged from the return air duct of the forced air system, said purification unit comprising:
a control circuit positioned within said housing;
an actuator coupled to said housing, said actuator being electrically coupled to said control circuit, said actuator being positioned on said front wall adjacent to said top side, said actuator being actuatable between an on position and an off position;
an ultraviolet light emitter positioned within an interior of said housing, said ultraviolet light emitter being positioned closer to said inlet opening than said outlet opening, said ultraviolet light emitter being electrically coupled to said actuator such that said ultraviolet light emitter is actuated and de-actuated by said actuator, said ultraviolet light emitter emitting electromagnetic radiation such that said ultraviolet light emitter is configured to urge air inwardly through said inlet opening and outwardly through said outlet opening via thermal convection, said ultraviolet light emitter being configured to eliminate bacteria in the air such that odors are eliminated from the air moving from said inlet opening to said outlet opening;
a motion sensor coupled to said housing, said motion sensor being positioned on said front wall such that said motion sensor is configured to detect motion proximate said housing, said motion sensor being electrically coupled to said ultraviolet light emitter, said motion sensor de-actuating said ultraviolet light emitter when said motion sensor detects motion, said motion sensor actuating said ultraviolet light emitter when said motion sensor ceases to detect motion; and
a power cord electrically coupled to said actuator, said power cord extending outwardly from said bottom side, said power cord having a distal end with respect to said bottom side, said distal end having a plug electrically coupled thereto, said plug being configured to be electrically coupled to a power source.
